(19) United States
(12) Patent Application Publication
(10) Pub. No.: US 2005/0054064 A1
(43) Pub. Date: Mar. 10, 2005

(54) Production of alcohol from a combination of sweet sorghum and other feedstock

(76) Inventors: Srikrishna Talluri, Southfield, MI (US); Palaniswamy Ramaswamy Aare, Hyderabad (IN)

Correspondence Address: SRIKRISHNA TALLURI, 23730 POND ROAD, # 121, SOUTHFIELD, MI, 48034, US

(21) Appl. No.: 10/935,885

(22) Filed: Sep. 8, 2004

Related U.S. Application Data
(60) Provisional application No. 60/501,068, filed on Sep. 8, 2003.

(51) Int. Cl. C12P 7/06; C12C 11/00

(52) U.S. Cl. 435/161; 426/11

(57) ABSTRACT

A method and system to produce alcohol from a combination of sweet sorghum and other agro-based feedstock is described. The base or major raw material is sweet sorghum and the agro feedstock may be molasses, cantaloupe fruits, mango, pineapple etc. Juice is extracted from sweet sorghum and the molasses (or juice from other feedstock) are mixed and heated to about 105 degree C. and then rapidly cooled down to about 30 degree C. when yeast is added and the fermentation takes place for about a period of 72 hours. Thereafter, the fermented juice is distilled to produce alcohol. The water consumed in this process is very less compared to traditional methods. Unique and distinct flavored alcohol/spirit can be produced without any special equipment or modifications to existing machinery. Damaged, over-ripen and unmarketable fruits may also be utilized for production of alcohol in combination with sweet sorghum juice.

### CROSS-REFERENCE TO RELATED APPLICATIONS

This application claims the benefit of priority of U.S. Provisional Patent Application Ser. No. 60/501,068, filed Sep. 8, 2003 and entitled “PRODUCTION OF ALCOHOL FROM A COMBINATION OF SWEET SORGHUM AND OTHER FEEDSTOCK”, the subject matter of which is hereby incorporated by reference herein.

### BACKGROUND OF THE INVENTION

This invention relates generally to alcohol production and, more specifically, to a novel method that uses a combination of sweet sorghum, other select crops and relevant industrial bi-products for the production of ethyl alcohol (ethanol).

ETHYL ALCOHOL PRODUCTION

Ethyl alcohol, also called ethanol (C2H5OH) is an oxygenated organic chemical compound that belongs to the alcohol family (—OH) and has numerous industrial and commercial applications. For instance, it is used in the manufacture of toiletries and cosmetics, inks and varnishes, in the production of numerous organic chemicals such as ethers and dienes as well as in the production of beverage alcohols. It is also used in combination with gasoline as fuel for automotive engines and by itself, has exhibited immense promise as a clean burning, low emission alternate fuel.

Ethyl alcohol is derived from two primary processes: hydration of ethylene and fermentation of sugars. All beverage alcohol and a good percentage of that used in the industry in obtained through fermentation of sugar crops (sugar cane, sugar beet etc.), grains (corn, barley etc.), potato mashes and sugar by-products such as molasses.

Fermentation involves the breaking down of complex organic substances into simpler ones. Alcohol fermentation is enabled by several types of bacteria and yeasts. Through a simple enzymatic anaerobic action, yeasts convert sugar molecules into alcohol and carbon dioxide.

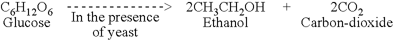

The above fermentation reaction is the basic method in the production of alcohol through fermentation. Glucose (a type of sugar) is broken down into ethyl alcohol and carbon dioxide by yeasts. The above reaction is a simplified one that actually involves intermediate steps—that of the generation of pyruvic acid, its conversion into acetaldehyde and finally to ethanol.

The above fermentation reaction generally yields less than 10% of ethanol. To obtain higher concentrations of alcohol, this ethanol is subjected to a distillation process.

MOLASSES: Molasses (also known as cane molasses in the U.S.A and as treacle in Britain) are a valued by-product of sugar production from Sugarcane (or Sugar beets) after the maximum amount of economically extractable sugar is removed from the raw material. It is a dark brown viscous liquid and generally contains 30%-50% of uncrystallized, fermentable sugars and some sucrose. They are often used as cattle-feed, as binding material for moulds and when refined, are used as a sweet syrup delicacy. Commercially, molasses are used in the manufacture of several chemical compounds and more importantly form the chief source (feedstock) for alcohol production in several countries.

However, the process of extracting alcohol from molasses has several disadvantages. This process results in a high level of effluents, which leads to a high level of air, water and soil pollution if left untreated, while the treatment of the same is a high capital cost implementation. Moreover, alcohol production from molasses requires a high amount of water (around four to five times by volume—as the actual amount of molasses used), which is a precious resource in several developing countries. The International Water Management Institute (IWMI) points out that an estimated 50 countries will face water severity in the next few years. Moreover, in some countries, the production of alcohol from molasses is not allowed during monsoon (rainy) season—to prevent and contain soil and water pollution due to effluents from this process.

Another major disadvantage is the scarcity of molasses both in terms of their year round unavailability and in the amount available for alcohol production. The quantity of molasses available therefore becomes a limiting factor in the actual amount of alcohol produced.

SWEET SORGHUM: Sweet sorghum [Sorghum bicolor (L.) Moench] is grown in different parts of the world, generally as a pure crop for commercial grain production. It has a marked resistance to drought, saline-alkaline soils and tolerance to waterlogging and is adaptable to a wide range of climates. Further, sweet sorghum requires only about a mere one-fourth of water content when compared to sugarcane.

Sweet sorghum is resourceful in that it contains high sugar percentages in the stem and provides the same for production of sugar, for paper manufacture, as fodder etc. Sorghum grain has also been used traditionally in making several food products like unleavened bread, cakes and malted beverages. Syrup made from sweet sorghum is used as a delicacy and for food flavoring. Sweet sorghum grains, flour and starch are also used for alcohol production. While sugar content in sweet sorghum is lower when compared to that of sugarcane or sugar beets new varieties/strains of sweet sorghum with comparable sugar levels are being developed. Further, sweet sorghum contains less sulphur content and hence produces more pure alcohol.

It is therefore desirable to solve current problems, overcome limitations in the prior art and increase alcohol production output.

### BRIEF SUMMARY OF THE INVENTION

Accordingly, it is an object of the present invention to provide a method and apparatus for alcohol production that utilizes a combination of sweet sorghum and molasses as feedstock.

It is another object of the present invention to provide a method and apparatus for alcohol production from a combination of sweet sorghum and other natural sources as feedstock.

It is yet another object of the present invention to provide a method and apparatus for alcohol production that uses sweet sorghum and finished sugar by-products as feedstock.

It is still yet another object of the present invention to provide a method and apparatus for alcohol production that overcomes feedstock shortages, enables stable year round production of alcohol, increases equipment utilization rate and delivers economic benefits.

It is still yet another object of the present invention to provide a method and apparatus for alcohol production that reduces the environmentally detrimental effluents from the alcohol production process.

It is still yet another object of the present invention to provide a method and apparatus for alcohol production that reduces the amount of water required per given amount of alcohol produced.

It is still yet another object of the present invention to provide a method and apparatus for alcohol production that utilizes (unmarketable) damaged and over ripe fruits as a feedstock, along with sweet sorghum for alcohol production.

### DETAILED DESCRIPTION OF THE INVENTION

The primary embodiment of the present invention overcomes the uncertainties in alcohol production due to feedstock shortages (scarcity of molasses) and addresses environment and water utilization issues by using sweet sorghum (which uses less water than other sugar yielding crops and is harvested three times per year and hence more prone to year round availability) as basic feedstock and adding of molasses later in the alcohol production process.

In the production of alcohol from Sweet Sorghum and molasses, Sweet Sorghum stems or stalks are crushed using commercially available cane crushers that may by two roller, three roller, five roller crushers or modern equipment to grind the stalks and extract the juice. A special arrangement is added to the crushing equipment to spray hot water at 90-99 degree Celsius on the crushing rollers in order to extract more sugars from the stalks. The filtered juice collected in the juice tank is transferred to an equipment called ‘Agitator’, which is commercially available in the market. This Agitator is equipped with motorized paddle, steam sparging arrangement and steam jacket for heating the juice. Enzymes (Beta Enzymes) are added first and the juice is heated up to 60 degree Celsius. Further (Alpha Enzymes and nutrients are added and heated up to 75 degree Celsius. With this Sweet Sorghum juice, molasses is added up to 30% v/v (volume/volume) and paddled in order to mix this molasses thoroughly to increase the sugar level in the juice up to 33% without addition of water. The resultant juice mixture has higher sugar content than from Sweet Sorghum alone while utilizing the Sweet Sorghum juice for liquefaction of molasses instead of doing separate liquefaction process by mixing water up to 4 times by v/v (molasses/water) with molasses for liquefaction process. This juice/syrup (now it is also called syrup) is subjected to heat at boiling temperature up to 105 degree Celsius in order to sterilize this syrup. The Ph of the juice is adjusted to 4.5-5 Ph. The vapor is routed to condensers in order to condense and condensate is collected and reused. As an increase in the sugar levels automatically results in higher output of alcohol such a mixer of molasses to the juice from Sweet Sorghum yields higher yield of alcohol.

The hot juice/syrup is rapidly cooled from about 105 degree Celsius to 30 degree Celsius. With the cooled syrup, distilleries yeast is added and subjected to fermentation either by ‘batch method’ process or ‘continuous fermentation’ method for which necessary equipment with cooling and temperature control arrangement (up to 32 degree Celsius) are commercially available in the market. The fermentation of juice may be allowed up to 72 hours, which is known, as Fermented wash/Wort shall contain up to 16% of alcohol v/v. The Wort is subjected to distillation (fraction distillation/multi pressure distillation) to produce alcohol.

This process utilizes lower water content (than used for molasses alone). This also results in lower effluent and reduces the associated effluent treatment cost.

In another embodiment of the present invention, Cashew Apple fruits (over-ripe, damaged and unmarketable fruits) are crushed and juice is extracted which contains 9-12% different fermentable sugars (Fructose, Sucrose etc.) and necessary nutrients that assists in growth of yeast. The extracted juice is treated with enzymes to convert the fructose, sucrose and other sugars into fermentable juice/syrup. The resultant syrup is added to the juice obtained from Sweet Sorghum processing to increase the sugar content up to 33%. The Ph of the juice is adjusted to 4.5-5 Ph. The mixture of juice/syrup is processed for fermentation and distillation to produce alcohol. The cashew apple also adds unique flavor to the alcohol/spirit, which is useful for beverage alcohol.

In another embodiment of present invention the juice extracted from Cashew Apple fruits is heated to the required temperature with enzymes in Agitator and molasses are then added up to 30% v/v with out any addition of water. This mixture is added along with juice obtained from the Sweet Sorghum processing to increases sugar content to up to 33% and entire juice is boiled up to 105 degree Celsius. The Ph of the juice is adjusted to 4.5-5 Ph. This mixture of juice/syrup is rapidly cooled to up to 20-30 degree Celsius. The resultant mixture of juice/syrup is subjected to fermentation and distillation to produce alcohol.

In another embodiment of present invention the juice extracted from the Sweet Sorghum is added with sugar powder, which is a byproduct of sugar mills. This resultant mixture will have sugars up to 33%. This juice is treated with enzymes is heated up to 105 degree celsius and is sterilized and rapidly cooled up to 20-30 degree celsius. The Ph of the juice is adjusted to 4.5-5 Ph. The cooled syrup is subjected to fermentation using distillery yeast. This fermented wash is distilled to produce alcohol. This alcohol has a distinct flavor, which is used for beverage alcohol.

In another embodiment of present invention the pulp/juice extracted from damaged, over-ripen and marketable/unmarketable mango fruits, after treated with enzymes to convert fructose, sucrose, glucose and other sugars into fermentable juice/syrup is added with the juice obtained from Sweet Sorghum processing with out any addition of water to increase sugar content up to 33%. This mixture is heated up to 105 degree Celsius. The Ph of the juice is adjusted up to 4.5-5 Ph. The juice/syrup is rapidly cooled to 20-30 degree Celsius. The mixture of juice/syrup is processed for fermentation and distillation to produce alcohol. Mango fruit also adds unique flavor to the alcohol/spirit (Useful for beverage alcohol).

In another embodiment of present invention the juice extracted from Mango fruits which contains 9-10% fermentable sugars like fructose, sucrose, glucose and other sugars in different form, is heated to the required temperature with enzymes in Agitator and molasses is added up to 30% v/v with out any addition of water. This mixture is added along with juice obtained from the Sweet Sorghum processing to increases sugars up to 33% and entire juice is boiled up to 105 degree Celsius. The Ph of the juice is adjusted to 4.5-5 Ph. This mixture of juice/syrup is rapidly cooled to 20-30 degree Celsius. The resultant mixture of juice/syrup is subjected to fermentation and distillation to produce alcohol.

In another embodiment of the present invention the pulp/juice extracted from damaged, over-ripen and marketable/unmarketable Pineapple fruits, (which contains necessary nutrients that assists in growth of yeast) after treated with enzymes to convert fructose, sucrose, glucose and other sugars into fermentable juice/syrup is added with the juice obtained from Sweet Sorghum processing without any addition of water to increase sugar content to 33% and entire juice is boiled up to 105 degree Celsius. The Ph of the juice is adjusted to 4.5-5 Ph. This mixture of juice/syrup is rapidly cooled to 20-30 degree Celsius. The mixture of juice/syrup is processed for fermentation and distillation to produce alcohol. Pineapple fruit also adds unique flavor to the alcohol/spirit (Useful for beverage alcohol).

In another embodiment of present invention the juice extracted from Pineapple fruits which contains 8-10% fermentable sugars like fructose, sucrose, glucose and other sugars in different form with necessary nutrients that assists in growth of yeast, is heated to the required temperature with enzymes in Agitator and molasses is added up to 30% v/v with out any addition of water. This mixture is added along with juice obtained from the Sweet Sorghum processing to increase sugars up to 33% and entire juice is boiled up to 105 degree Celsius. The Ph of the juice is adjusted to 4.5-5 Ph. This mixture of juice/syrup is rapidly cooled to 20-30 degree Celsius. The resultant mixture of juice/syrup is subjected to fermentation and distillation to produce alcohol.

In another embodiment of present invention the pulp/juice extracted from damaged, over-ripen and marketable/unmarketable Cantaloupe fruits, after treated with enzymes to convert fructose, sucrose, glucose and other sugars into fermentable juice/syrup is added with the juice obtained from Sweet Sorghum processing with out any addition of water to increase the sugar content up to 33% and entire juice is boiled up to 105 degree Celsius. The Ph of the juice is adjusted to 4.5-5 Ph. This mixture of juice/syrup is rapidly cooled up to 20-30 degree Celsius. The mixture of juice/syrup is processed for fermentation and distillation to produce alcohol. Cantaloupe fruits also add unique flavor to the alcohol/spirit (Useful for beverage alcohol).

In another embodiment of present invention the juice extracted from Cantaloupe fruits which contains 8-10% fermentable sugars like fructose, sucrose, glucose and other sugars in different form, is heated to the required temperature with enzymes in Agitator and molasses is added up to 30% v/v with out any addition of water. This mixture is added along with juice obtained from the Sweet Sorghum processing to increases sugars up to 33% and entire juice is boiled up to 105 degree Celsius. The Ph of the juice is adjusted to 4.5-5 Ph. This mixture of juice/syrup is rapidly cooled to 20-30 degree Celsius. The resultant mixture of juice/syrup is subjected to fermentation and distillation to produce alcohol.

In another embodiment of present invention the pulp extracted from damaged, over-ripen and marketable/unmarketable Banana fruits, which contains 11-17% sugars is added with the juice obtained from Sweet Sorghum processing without any addition of water up to 30% to increase sugars up to 33%. This mixture is treated with enzymes to convert fructose, sucrose, other sugars and starch into fermentable sugars and entire juice is boiled up to 105 degree Celsius. The Ph of the juice is adjusted up to 4.5-5 Ph. This mixture of juice/syrup is rapidly cooled to 20-30 degree Celsius. The mixture of juice/syrup is processed for fermentation and distillation to produce alcohol. Banana fruits also adds unique flavor to the alcohol/spirit (Useful for beverage alcohol).

In another embodiment of the present invention, dried Mahua flowers (Madhuca latifolia and Madhuca longifolia (Koenig) Macbrm; Synonyms Bassia Longifolia, Illipe longifoila) which contains sugars up to 60-72% are crushed and water is added to convert into juice. The juice is treated with enzymes to convert the fructose, sucrose and other sugars into fermentable sugars. The syrup/juice contains necessary nutrients that assist in growth of yeast. The resultant juice/syrup is added with the obtained from Sweet Sorghum processing to increase the sugar content up to 33% and entire juice is boiled up to 105 degree Celsius. The Ph of the juice is adjusted to 4.5-5Ph. This mixture of juice/syrup is rapidly cooled to 20-30 degree Celsius. The mixture of juice/syrup is processed for fermentation and distillation to produce alcohol. The Mahua Flowers also adds unique flavor to the alcohol/spirit (Useful for beverage alcohol).

In another embodiment of present invention the juice extracted from Mahua Flowers is heated to the required temperature with Enzymes in Agitator and molasses is added up to 30% v/v. This mixture is added along with juice obtained from the Sweet Sorghum processing to increases sugar up to 33% and entire juice is boiled up to 105 degree Celsius. The Ph of the juice is adjusted to 4.5-5 Ph. This mixture of juice/syrup is rapidly cooled to 20-30 degree Celsius. The resultant mixture of juice/syrup is subjected to fermentation and distillation to produce alcohol.

In another embodiment of the present invention, Date fruits (without seeds), which contain sugars up to 72%, are crushed and water is added to convert into juice. The juice is treated with enzymes to convert the fructose, sucrose and other sugars into fermentable juice/syrup. The syrup/juice contains necessary nutrients that assist in growth of yeast. The resultant syrup added to the juice obtained from Sweet Sorghum processing to increase the sugar content up to 33% and entire juice is boiled up to 105 degree Celsius. The Ph of the juice is adjusted to 4.5-5 Ph. This mixture of juice/syrup is rapidly cooled to 20-30 degree Celsius. The mixture of juice/syrup is processed for fermentation and distillation to produce alcohol. The Date fruit also adds unique flavor to the alcohol/spirit (Useful for beverage alcohol).

In another embodiment of the present invention, Tapioca/Cassava chips, which contain Starch, are cooked in water with enzymes to break into fermentable sugars. This fermentable juice/syrup is added to the juice obtained from Sweet Sorghum processing to increase the sugar content up to 33% and entire juice is boiled up to 105 degree Celsius. The Ph of the juice is adjusted to 4.5-5 Ph. This mixture of juice/syrup is rapidly cooled up to 20-30 degree Celsius. The mixture of juice/syrup is processed for fermentation and distillation to produce alcohol.

In another embodiment of the present invention, Sweet sorghum grain is milled and with addition of water boiled in jet cooker (liquefaction) and treated with Enzymes to convert the starch into fermentable sugars (saccharification). This syrup is added to the juice obtained from Sweet Sorghum processing up to 30% v/v to increase the sugar content up to 33% and entire juice is boiled up to 105 degree Celsius. The Ph of the juice is adjusted to 4.5-5 Ph. This mixture of juice/syrup is rapidly cooled up to 20-30 degree Celsius. The mixture of juice/syrup is processed for fermentation and distillation to produce alcohol. The alcohol from this process will have unique flavor to the alcohol/spirit (Useful for beverage alcohol).

In another embodiment of the present invention, Sweet sorghum grain is milled and with addition of water boiled in jet cooker (liquefaction) and treated with Enzymes to convert the starch into fermentable sugars (saccharification). This syrup is cooled up to 50-55 degree Celsius and dried Sweet Sorghum Malt is added up to 20% to the syrup and maintained in the same temperature for a certain length of time (30-45 minutes). This mash is added to the juice obtained from the Sweet Sorghum process and the entire juice is boiled up to 65 degree Celsius to increase sugar contents up to 33%. The Ph of the juice is adjusted up to 4.5-5 Ph. This mixture of juice/syrup is rapidly cooled up to 20-30 degree Celsius. The mixture of juice/syrup is processed for fermentation. The fermented mash is treated to remove solids using centrifuge machines (in these process yeast is also separated from fermented mash) and distilled to produce alcohol. The malt and grain both will add unique and distinct flavor to the alcohol/spirit (Useful for beverage alcohol).

In another embodiment of the present invention, Sweet sorghum malt syrup (Dried Sweet Sorghum malt is converted into liquid syrup by liquefaction and saccharification). This malt syrup is added to the juice obtained from the Sweet Sorghum Process and the entire juice is boiled up to 65 degree Celsius to increase sugar content up to 33%. The Ph of the juice is adjusted up to 4.5-5 Ph. This mixture of juice/syrup is rapidly cooled up to 20-30 degree Celsius. The mixture of juice/syrup is processed for fermentation and distillation to produce alcohol. The malt also adds unique and distinct flavor to the alcohol/spirit (Useful for beverage alcohol).

In another embodiment of the present invention, Barley grain is milled and with addition of water boiled in jet cooker (liquefaction) is treated with enzymes to convert the starch into fermentable sugars (saccharification). This syrup is cooled to 50-55 degree Celsius and dried Barley Malt is added up to 20% to the syrup and maintained in the same temperature for a certain length of time (3045 minutes). This mash is added to the juice obtained from the Sweet Sorghum process and the entire juice is boiled up to 65 degree Celsius to increase sugar content up to 33%. The Ph of the juice is adjusted up to 4.5-5 Ph. This mixture of juice/syrup is rapidly cooled up to 20-30 degree Celsius. The mixture of juice/syrup is processed for fermentation. The fermented mash is treated to remove solids using centrifuge machines (in these process yeast is also separated from Fermented mash) and distilled to produce alcohol. The Barley malt and grain both will add unique and distinct flavor to the alcohol/spirit (Useful for beverage alcohol).

In another embodiment of the present invention, Barley malt syrup (Dried Barley malt is converted into liquid syrup by liquefaction and saccharification). This malt syrup is added to the juice obtained from the Sweet Sorghum Process and the entire juice is boiled up to 65 degree Celsius to increase the sugar content up to 33%. The Ph of the juice is adjusted to 4.5-5 Ph. This mixture of juice/syrup is rapidly cooled to 20-30 degree Celsius. The mixture of juice/syrup is processed for fermentation and distillation to produce alcohol. The malt also adds unique and distinct flavor to the alcohol/spirit (Useful for beverage alcohol).

In another embodiment of the present invention, a mixture of wheat, rice and corn grains are milled together or separately milled and mixed before liquefaction and saccharification. The mixture of milled coarse grains is cooked in jet cooker (pressurized cooker) and treated with Enzymes to convert the carbohydrates into fermentable sugars (liquefaction and saccharification). This syrup up to 40% v/v is added with the juice obtained from the Sweet Sorghum process (up to 60%) and entire juice is boiled up to 105 degree Celsius to increase the sugar content up to 33%. The Ph of the juice is adjusted to 4.5-5 Ph. This mixture of juice/syrup is rapidly cooled to 20-30 degree Celsius. The mixture of juice/syrup is processed for fermentation. The fermented mash is treated to remove solids using centrifuge machines or solid separators, which are commercially available in market (in these process yeast is also separated from fermented mash) and distilled to produce alcohol. The grain also adds unique and distinct flavor to the alcohol/spirit (Useful for beverage alcohol).

In above description the word Molasses can be construed to represent molasses derived from either Sugarcane or Sweet Sorghum or a mixture of both.

1. A method for the production of alcohol from a combination of sweet sorghum and other agro feedstock, wherein the base or major raw material is sweet sorghum.

2. A method of claim 1, where the agro feedstock added to sweet sorghum is molasses.

3. The process of claim 1 wherein the base is sweet sorghum and the agro feedstock is molasses, and wherein the sweet sorghum juice and the molasses are mixed and heated to about 105 degree C. and then rapidly cooled down to about 30 degree C. when yeast is added and the fermentation takes place for about a period of 72 hours, where after the fermented juice is distilled to produce alcohol.

4. A system for the production of alcohol from a combination of sweet sorghum and other agro based feedstock, wherein the base or major raw material is sweet sorghum.
